(19) United States
(12) Patent Application Publication
(10) Pub. No.: US 2024/0415755 A1
(43) Pub. Date: Dec. 19, 2024

(54) WHITENING COMPOSITION INCLUSION SOLUTION, PREPARATION METHOD THEREFOR, AND USE THEREOF

(71) Applicants: Guangzhou Fanzhirong Cosmetics Co., LTD., Guangzhou (CN); Guangzhou Qingnang Biotechnology Co., LTD, Guangzhou (CN)

(72) Inventors: Lushi CHENG, Guangzhou (CN); Anning WANG, Guangzhou (CN); Anzhang LI, Guangzhou (CN)

(73) Assignees: Guangzhou Fanzhirong Cosmetics Co., LTD., Guangzhou (CN); Guangzhou Qingnang Biotechnology Co., LTD, Guangzhou (CN)

(21) Appl. No.: 18/820,101

(22) Filed: Aug. 29, 2024

Related U.S. Application Data
(63) Continuation of application No. PCT/CN2024/085144, filed on Apr. 1, 2024.

(30) Foreign Application Priority Data
Mar. 31, 2023 (CN) 202310337523.4

(51) Int. Cl. A61K 8/49; A61K 8/14; A61K 8/34; A61K 8/37; A61K 8/55; A61K 8/64; A61K 8/67; A61K 8/9789; A61Q 19/02

(52) U.S. Cl. CPC A61K 8/498; A61K 8/14; A61K 8/345; A61K 8/375; A61K 8/553; A61K 8/64; A61K 8/676; A61K 8/9789; A61Q 19/02; A61K 2800/21

(57) ABSTRACT

A whitening composition comprising a bilayer vesicular inclusion with a skin cell membrane-like structure formed by a phospholipid, a polyol, a co-emulsifier, and a liquid oil, wherein the phospholipid serves as a main wall material of the inclusion, and an alcohol-soluble main ingredient, an auxiliary ingredient, an antioxidant ingredient, and an anti-inflammatory ingredient in an aqueous plant extract solution are encapsulated in the inclusion, with a signal molecule attached to the surface of the inclusion. The whitening composition inclusion solution of the present application has good whitening efficacy due to the synergistic effect among various components.

### CROSS-REFERENCE TO RELATED APPLICATIONS

The present application claims priority and benefit to the Chinese Patent Application No. 202310337523.4 filed with National Intellectual Property Administration, PRC on Mar. 31, 2023, the disclosure of which is incorporated herein by reference in its entirety.

### TECHNICAL FIELD

The present invention relates to the field of whitening skincare products, in particular to a whitening composition inclusion solution, a preparation method therefor, and use thereof.

### BACKGROUND

Glabridin (a flavonoid substance) is a natural plant extract extracted from polyphenols in wild Glycyrrhiza glabra in Africa. Glabridin has many effects such as antioxidant, immunomodulatory, anti-inflammatory, and antidepressant properties. Studies have shown that glabridin has anti-free radical activity and can effectively inhibit the damage caused by free radicals to cells.

The targets of action of glabridin and other common whitening agents are positioned in the cell nucleus of melanocytes. After an α-MSH signal molecule binds to an MC1-R receptor on the melanocyte membrane, resulting in intracellular reactions such as up-regulation of protein kinase A expression, the activated tyrosinase catalyzes tyrosine to ultimately form melanin. When inflammation such as allergy, acne, or sunburn occurs, the binding of α-MSH to the MC1-R receptor is also induced, so that the expression of inflammatory factors such as IL-1, IL-6, IL-8, and TNF-α is up-regulated, ultimately resulting in pigmentation after the inflammation heals. Therefore, the formation of melanin is caused by a series of complicated reactions within melanocytes, and in order to achieve a good whitening effect, it is necessary to consider from different perspectives in addition to the inhibition of tyrosinase activity.

The existing technical solutions are as follows: 1 In CN108451837A, functional ingredients are similarly compounded and encapsulated according to different whitening mechanisms, so that the effects of multi-target whitening, convenience in formula application, and the like are achieved. (2) In CN114796008A, glabridin and polypeptide are simultaneously encapsulated. 3 CN115778886A discloses a glabridin plant-derived micro inclusion and a preparation method therefor. However, the inventors of the present invention have found that the products obtained by the above technical solutions in the prior art have problems such as poor stability (for example, the products turn pink or yellow after being left to stand at room temperature for 30 days) and poor whitening effect.

Therefore, there is still an urgent need for a whitening product with good stability and excellent whitening effect.

### SUMMARY

In view of the above problems, the present invention aims to provide a whitening composition inclusion solution with better whitening efficacy, a preparation method therefor, and use thereof.

In order to achieve the technical objective, in a first aspect, the present invention provides a preparation method for a nano-scale bilayer inclusion, comprising the following specific steps:
S1, weighing 1-3% of an alcohol-soluble main ingredient and 10-50% of a polyol by mass percentage, mixing the same in a water bath at 65° C., and stirring until complete dissolution is achieved to obtain a phase A;
S2, weighing 3-15% of a phospholipid, 1-3% of a co-emulsifier, 1-5% of a liquid oil, and 0.01-0.5% of a signal molecule by mass percentage, mixing the same in a water bath at 70° C., and stirring until complete dissolution is achieved to obtain a phase B;
S3, preparing an aqueous plant extract solution, then weighing 0.01-2% of an auxiliary ingredient and 0.01-4% of an antioxidant ingredient by mass percentage, followed by dissolving in 17.5-83.97% of the aqueous plant extract solution, and heating to 65° C. to obtain a phase C;
S4, mixing the phase A with the phase B, followed by stirring uniformly, and then adding the phase C for emulsification to obtain a coarse emulsion; and
S5, subjecting the coarse emulsion to a high-pressure shearing treatment at a temperature of 30-40° C. to obtain a nano-scale inclusion, thus ultimately obtaining a whitening composition inclusion solution.

Preferably, in step S4, an emulsifying machine is used with an output power of 0.2 kW, a rotation speed of 3000-8000 rpm, and a high-speed shearing dispersion time of 3-10 min;

the shearing is performed at a pressure of 400-1000 bar, and the number of cycles of shearing is 5-10.

Preferably, the aqueous plant extract solution is an aqueous extract solution of a herbal anti-inflammatory plant, the herbal anti-inflammatory plant being one or a combination of two or more of Paeonia suffruticosa flower, Paeonia suffruticosa root, Rosa rugosa flower, and Portulaca oleracea.

Preferably, in step S3, the herbal anti-inflammatory plant is washed, dried, and crushed to obtain a plant powder, and the plant powder is decocted with water in a mass ratio of 1:10-1:20 under reflux at 50-80° C. for 8-24 h to obtain a decoction solution; then the decoction solution is filtered, and a filtrate is treated with a macroporous anion exchange resin in a volume ratio of (10-15):1, with a retention time of 15-25 min, to obtain a purified aqueous plant extract solution.

Preferably, the alcohol-soluble main ingredient is glabridin, the auxiliary ingredient is glutathione, and the signal molecule is palmitoyl tripeptide-8.

Preferably, the polyol is one or a combination of two or more of glycerol, propylene glycol, butylene glycol, dipropylene glycol, pentylene glycol, and isoprene glycol;
the co-emulsifier is one or a combination of two or more of polyglycerol-10 stearate, polyglyceryl-10 laurate, polyglyceryl-10 oleate, ethoxylated hydrogenated castor oil, and potassium cetyl phosphate;
the liquid oil is one or a combination of two or more of caprylic/capric triglyceride, Limnanthes alba seed oil, squalane, olive oil, and Paeonia suffruticosa seed oil;
the phospholipid is one or a combination of two or more of soybean lecithin, hydrogenated lecithin, and soybean phosphatidylcholine;
the antioxidant ingredient is one or a combination of two or more of vitamin C, 3-o-ethyl ascorbic acid, ascorbyl glucoside, magnesium ascorbyl phosphate, sodium ascorbyl phosphate, and ascorbic acid polypeptide.

Preferably, a formulation ratio of the whitening composition inclusion solution is: 1.1±0.1% of the glabridin, 20±5% of the polyol, 6±2% of the phospholipid, 2±0.5% of the co-emulsifier, 2±0.5% of the liquid oil, 0.15±0.05% of the palmitoyl tripeptide-8, 1±0.2% of the glutathione, 3±0.5% of the antioxidant ingredient, and 60±15% of the aqueous plant extract solution by mass percentage.

In some embodiments, the nano-scale bilayer inclusion prepared by using the preparation method described above has a particle size of 20-110 nm. In some embodiments, the nano-scale bilayer inclusion prepared by using the preparation method described above has a particle size of 30-110 nm. In some embodiments, the nano-scale bilayer inclusion prepared by using the preparation method described above has a particle size of 30 nm, 80 nm, or 110 nm.

In a second aspect, the present invention provides a whitening composition inclusion solution obtained by the preparation method according to the first aspect.

Provided is a high-transparency and stable whitening composition inclusion solution prepared by using the preparation method for the nano-scale bilayer inclusion according to the first aspect, wherein the phospholipid serves as a main wall material of the inclusion, and the alcohol-soluble main ingredient, the auxiliary ingredient, the antioxidant ingredient, and the plant extract comprising an anti-inflammatory ingredient are encapsulated in the inclusion, with the signal molecule attached to the surface of the inclusion.

In a third aspect, the present invention provides use of the aforementioned whitening composition inclusion solution.

Provided is use of the whitening composition inclusion solution according to the second aspect in the field of cosmetics.

The present invention has the following beneficial effects: The whitening composition inclusion solution of the present application can achieve a good whitening effect due to the relatively good synergistic effect among various components. Meanwhile, the whitening composition inclusion solution of the present application is an inclusion containing a bimolecular vesicular structure, has good stability, and can stably encapsulate the effective ingredients therein while also being easily absorbed by the skin. Traditionally, palmitoyl tripeptide-8 is used as a polypeptide for relieving and resisting allergy (and the palmitoyl tripeptide-8 also has an α-MSH biomimetic effect). However, in the present application, the palmitoyl tripeptide-8 is used as a target peptide, and due to its amphiphilicity, it can effectively attach to the surface of the inclusion to form a target, wherein the hydrophilic group of the palmitoyl tripeptide-8 can bind to an MC1-R receptor of a melanocyte; and at the same time, the glabridin and the glutathione are used for compounding to generate a good synergistic effect, so that the generation of eumelanin is effectively reduced, thereby further improving the whitening efficacy. The formulation ratio of each ingredient in the whitening composition inclusion solution of the present application is strictly selected, which has a relatively significant influence on the exertion of the whitening efficacy.

In a fourth aspect, the present invention provides a whitening composition inclusion solution.

Provided is a whitening composition inclusion solution, having a preparation method comprising:
(1) based on mass ratio, mixing 1% of glabridin with 20% of butylene glycol in a water bath at 65° C., and stirring until complete dissolution is achieved to obtain a phase A;
(2) based on mass ratio, mixing 2% of polyglycerol-10 laurate, 1.5% of Paeonia suffruticosa seed oil, 5% of soybean lecithin, and 0.15% of palmitoyl tripeptide-8 in a water bath at 70° C., and stirring until complete dissolution is achieved to obtain a phase B;
(3) based on mass ratio, dissolving 1% of glutathione and 3% of 3-O-ethyl ascorbic acid in 66.35% of an aqueous solution of Rosa rugosa flower, and heating to 65° C. to obtain a phase C,
wherein the preparation of the aqueous solution of Rosa rugosa flower comprising: washing, drying, and crushing Rosa rugosa flower as a herbal anti-inflammatory plant to obtain a plant powder, decocting the plant powder with water in a mass ratio of 1:15 under reflux at 60° C. for 8 h to obtain a decoction solution, then filtering the decoction solution, and treating a filtrate with a macroporous anion exchange resin in a volume ratio of 10:1, with a retention time of 20 min, to obtain a purified aqueous plant extract solution;
(4) mixing the phase A and the phase B with stirring, and then adding the phase C for emulsification to obtain a coarse inclusion emulsion; and
(5) subjecting the coarse emulsion to a high-pressure shearing treatment to achieve a nano-scale size, with a circulating water bath controlled at 30-40° C. and 6 cycles of shearing under a pressure of 800 bar, to obtain the whitening composition inclusion solution.

In some embodiments, the whitening composition inclusion solution has a zeta potential of −34 m V.

In some embodiments, the inclusion in the whitening composition inclusion solution has a particle size of 30 nm.

### DETAILED DESCRIPTION OF THE EMBODIMENTS

The present invention will be further illustrated in detail with reference to the following specific examples.

The specific examples of the present invention are as follows: Provided is a preparation method for a whitening composition inclusion solution, comprising the following specific steps:

In S101, glabridin and a polyol are proportionally mixed in a water bath at 65° C., and the mixture is stirred until complete dissolution is achieved to obtain a phase A.

In S102, a co-emulsifier, a liquid oil, a phospholipid, and palmitoyl tripeptide-8 are proportionally mixed in a water bath at 70° C., and the mixture is stirred until complete dissolution is achieved to obtain a phase B.

In S103, an aqueous plant extract solution is prepared as follows: a herbal anti-inflammatory plant is washed, dried, and crushed to obtain a plant powder, and the plant powder is decocted with water in a mass ratio of 1:10-1:20 under reflux at 50-80° C. for 8-24 h to obtain a decoction solution; then the decoction solution is filtered, and a filtrate is treated with a macroporous anion exchange resin in a volume ratio of (10-15):1, with a retention time of 15-25 min, to obtain a purified aqueous plant extract solution; the aqueous plant extract solution is an aqueous extract solution of the herbal anti-inflammatory plant, the herbal anti-inflammatory plant being one or a combination of two or more of Paeonia suffruticosa flower, Paeonia suffruticosa root, Rosa rugosa flower, and Portulaca oleracea.

In S104, glutathione and an antioxidant ingredient are proportionally dissolved in the aqueous plant extract solution, and the mixture is heated to 65° C. to obtain a phase C.

In S105, the phase A and the phase B are proportionally mixed, followed by stirring uniformly, and then the phase C is added for emulsification to obtain a coarse emulsion.

In S106, the coarse emulsion is subjected to a high-pressure shearing treatment at a temperature of 30-40° C. to achieve a nano-scale size, wherein an emulsifying machine is used with an output power of 0.2 kW, a rotation speed of 3000-8000 rpm, and a high-speed shearing dispersion time of 3-10 min; the shearing is performed at a pressure of 400-1000 bar, and the number of cycles of shearing is 5-10. Finally, a high-transparency and stable whitening composition inclusion solution is obtained.

Provided is use of the whitening composition inclusion solution. The whitening composition inclusion solution can serve as a starting material for cosmetics. Typically, the mass ratio of the whitening composition inclusion solution in a cosmetic is 0.5-10%, and the whitening composition inclusion solution can be formulated into emulsions, creams, gels, and other dosage forms of cosmetics for use.

Provided is a whitening composition inclusion solution, comprising a bilayer vesicular inclusion with a skin cell membrane-like structure formed by a phospholipid, a polyol, a co-emulsifier, and a liquid oil, wherein the phospholipid serves as a main wall material of the inclusion, and glabridin (an alcohol-soluble main ingredient), glutathione (an auxiliary ingredient), an antioxidant ingredient, and an anti-inflammatory ingredient in an aqueous plant extract solution are encapsulated in the inclusion, with palmitoyl tripeptide-8 (a signal molecule) attached to the surface of the inclusion;
the whitening composition inclusion solution comprises: 1-3% of the glabridin, 10-50% of the polyol, 3-15% of the phospholipid, 1-3% of the co-emulsifier, 1-5% of the liquid oil, 0.01-0.5% of the palmitoyl tripeptide-8, 0.01-2% of the glutathione, 0.01-4% of the antioxidant ingredient, and 17.5-83.97% of the aqueous plant extract solution by mass percentage.

The polyol is one or a combination of two or more of glycerol, propylene glycol, butylene glycol, dipropylene glycol, pentylene glycol, and isoprene glycol; the co-emulsifier is one or a combination of two or more of polyglycerol-10 stearate, polyglyceryl-10 laurate, polyglyceryl-10 oleate, ethoxylated hydrogenated castor oil, potassium cetyl phosphate, and potassium phosphate; the liquid oil is one or a combination of two or more of caprylic/capric triglyceride, Limnanthes alba seed oil, squalane, olive oil, and Paeonia suffruticosa seed oil; the phospholipid is one or a combination of two or more of soybean lecithin, hydrogenated lecithin, and soybean phosphatidylcholine; the antioxidant ingredient is one or a combination of two or more of vitamin C, 3-o-ethyl ascorbic acid, ascorbyl glucoside, magnesium ascorbyl phosphate, sodium ascorbyl phosphate, and ascorbic acid polypeptide.

The glabridin, which has a resorcinol structure, holds potential value for its whitening efficacy, but is difficult to be applied in formulations due to its poor solubility in both water and oil. Meanwhile, the glabridin is prone to oxidation and discoloration when exposed to light, requiring techniques to improve its stability.

The technical principle of the present application is as follows: The inclusion uses the palmitoyl tripeptide-8 as a target peptide, and the hydrophilic group of the peptide exposed and embedded in the inclusion serves as a signal molecule (the hydrophilic group of the peptide exposed in the aqueous phase serves as a targeting signal molecule of the inclusion to bind to an MC1-R receptor), so that the affinity of the inclusion for melanocytes can be effectively increased, thereby improving the efficacy of the composition.

Meanwhile, by compounding with the glutathione, the glabridin has further improved whitening efficacy: the main whitening mechanism of the glabridin is to inhibit the activity of tyrosinase, thereby inhibiting the reaction of melanin production. Glutathione: melanin is divided into eumelanin (black to dark brown pigment) and pheomelanin (reddish-brown to yellow) as well as other less common types; the melanin in human epidermis mainly consists of a large portion of eumelanin and a small portion of pheomelanin; in the presence of glutathione, dopaquinone, which is formed from tyrosine catalyzed by tyrosinase, is partially synthesized into pheomelanin, with the rest majority being synthesized into eumelanin; by artificially increasing the concentration of glutathione, more dopaquinone is directed to form lighter-colored pheomelanin, thereby reducing the production of darker-colored eumelanin. Vitamin C or a vitamin C derivative: antioxidant, reducing already formed melanin into a colorless structure. Aqueous plant extract solution: anti-inflammatory.

An emulsifier with a critical packing parameter in the range of ½-1, such as soybean lecithin or hydrogenated lecithin, is selected as the main wall material for the inclusion, so as to form a bilayer vesicle with a skin cell membrane-like structure, such that the water-soluble active substances can be encapsulated in the inner aqueous phase, which also enhances penetration.

### Example 1

(1) Based on mass ratio, 2% of glabridin was mixed with 50% of glycerol in a water bath at 65° C., and the mixture was stirred until complete dissolution was achieved to obtain a phase A.

(2) Based on mass ratio, 3% of polyglycerol-10 stearate, 2% of Limnanthes alba seed oil, 10% of hydrogenated lecithin, and 0.05% of palmitoyl tripeptide-8 were mixed in a water bath at 70° C., and the mixture was stirred until complete dissolution was achieved to obtain a phase B.

(3) Based on mass ratio, 1% of glutathione and 2% of ascorbyl glucoside were dissolved in 29.95% of an aqueous solution of Paeonia suffruticosa root (obtained through preparation according to step S103, in which the aqueous plant extract solution was prepared as follows: a herbal anti-inflammatory plant was washed, dried, and crushed to obtain a plant powder, and the plant powder was decocted with water in a mass ratio of 1:15 under reflux at 60° C. for 8 h to obtain a decoction solution; then the decoction solution was filtered, and the filtrate was treated with a macroporous anion exchange resin in a volume ratio of 10:1, with a retention time of 20 min, to obtain the purified aqueous plant extract solution), and the resulting solution was heated to 65° C. to obtain a phase C.

(4) The phase A and the phase B were mixed with stirring, and then the phase C was added for emulsification to obtain a coarse inclusion emulsion.

(5) The coarse emulsion was subjected to a high-pressure shearing treatment to achieve a nano-scale size, with a circulating water bath controlled at 30-40° C. and 6 cycles of shearing under a pressure of 800 bar, to obtain a whitening composition inclusion solution having a zeta potential of −28 mV, wherein the inclusion had a particle size of 80 nm.

### Example 2

(1) Based on mass ratio, 3% of glabridin was mixed with 30% of dipropylene glycol in a water bath at 65° C., and the mixture was stirred until complete dissolution was achieved to obtain a phase A.

(2) Based on mass ratio, 3% of polyglycerol-10 oleate, 2% of caprylic/capric triglyceride, 8% of soybean lecithin, and 0.1% of palmitoyl tripeptide-8 were mixed in a water bath at 70° C., and the mixture was stirred until complete dissolution was achieved to obtain a phase B.

(3) Based on mass ratio, 1.5% of glutathione and 3% of ascorbyl glucoside were dissolved in 49.4% of an aqueous solution of Paeonia suffruticosa root (obtained through preparation according to step S103, in which the formulation ratio was the same as in Example 1), and the resulting solution was heated to 65° C. to obtain a phase C.

(4) The phase A and the phase B were mixed with stirring, and then the phase C was added for emulsification to obtain a coarse inclusion emulsion.

(5) The coarse emulsion was subjected to a high-pressure shearing treatment to achieve a nano-scale size, with a circulating water bath controlled at 30-40° C. and 6 cycles of shearing under a pressure of 800 bar, to obtain a whitening composition inclusion solution having a zeta potential of −22 mV, wherein the inclusion had a particle size of 110 nm.

### Example 3

(1) Based on mass ratio, 1% of glabridin was mixed with 20% of butylene glycol in a water bath at 65° C., and the mixture was stirred until complete dissolution was achieved to obtain a phase A.

(2) Based on mass ratio, 2% of polyglycerol-10 laurate, 1.5% of Paeonia suffruticosa seed oil, 5% of soybean lecithin, and 0.15% of palmitoyl tripeptide-8 were mixed in a water bath at 70° C., and the mixture was stirred until complete dissolution was achieved to obtain a phase B.

(3) Based on mass ratio, 1% of glutathione and 3% of 3-O-ethyl ascorbic acid were dissolved in 66.35% of an aqueous solution of Rosa rugosa flower (obtained through preparation according to step S103, in which the formulation ratio was the same as in Example 1), and the resulting solution was heated to 65° C. to obtain a phase C.

(4) The phase A and the phase B were mixed with stirring, and then the phase C was added for emulsification to obtain a coarse inclusion emulsion.

(5) The coarse emulsion was subjected to a high-pressure shearing treatment to achieve a nano-scale size, with a circulating water bath controlled at 30-40° C. and 6 cycles of shearing under a pressure of 800 bar, to obtain a whitening composition inclusion solution having a zeta potential of −34 mV, wherein the inclusion had a particle size of 30 nm. In Example 3, the effective ingredients encapsulated were in appropriate amounts, and the particle size of the inclusion was moderate.

The preparation methods of Examples 1-3 could all be used to obtain a stable whitening composition inclusion solution. Additionally, after being left to stand for 30 days, the whitening composition inclusion solutions obtained in Examples 1-3 were able to maintain the appearance with high transparency and slight blue tint, remaining homogeneous and non-agglomerated. Due to the relatively high content of the main active ingredient glabridin encapsulated in Examples 1 and 2, the particle size of the circular vesicles formed became larger with the increase in the content of the ingredient, so that the whitening composition inclusion solution obtained in Example 2 had the largest particle size, followed by Example 1 with the second largest particle size, and Example 3 with the smallest particle size. Since the smaller the particle size of the inclusion, the easier it is to be absorbed transdermally through the intercellular pathways of the skin, in order to verify the efficacy of the composition prepared under the optimal conditions of the preparation method, Example 3 was selected for subsequent comparison with the comparative examples.

### Comparative Example 1: Without Addition of Palmitoyl Tripeptide-8

(1) Based on mass ratio, 1% of glabridin was mixed with 20% of butylene glycol in a water bath at 65° C., and the mixture was stirred until complete dissolution was achieved to obtain a phase A.

(2) Based on mass ratio, 2% of polyglycerol-10 laurate, 1.5% of Paeonia suffruticosa seed oil, and 5% of soybean lecithin were mixed in a water bath at 70° C., and the mixture was stirred until complete dissolution was achieved to obtain a phase B.

(3) Based on mass ratio, 1% of glutathione and 3% of 3-o-ethyl ascorbic acid were dissolved in 66.5% of an aqueous solution of Paeonia suffruticosa flower (obtained through preparation according to step S103, in which the formulation ratio was the same as in Example 1), and the resulting solution was heated to 65° C. to obtain a phase C.

(4) The phase A and the phase B were mixed with stirring, and then the phase C was added for emulsification to obtain a coarse inclusion emulsion.

(5) The coarse emulsion was subjected to a high-pressure shearing treatment to achieve a nano-scale size, with a circulating water bath controlled at 30-40° C. and 6 cycles of shearing under a pressure of 800 bar, to obtain a whitening composition inclusion solution having a zeta potential of −34 mV, wherein the inclusion had a particle size of 30 nm. In Comparative Example 1, palmitoyl tripeptide-8 was not added.

### Comparative Example 2: Without Addition of Co-Emulsifier

(1) Based on mass ratio, 1% of glabridin was mixed with 20% of butylene glycol in a water bath at 65° C., and the mixture was stirred until complete dissolution was achieved to obtain a phase A.

(2) Based on mass ratio, 1.5% of Paeonia suffruticosa seed oil, 5% of soybean lecithin, and 0.15% of palmitoyl tripeptide-8 were mixed in a water bath at 70° C., and the mixture was stirred until complete dissolution was achieved to obtain a phase B.

(3) Based on mass ratio, 1% of glutathione and 3% of 3-O-ethyl ascorbic acid were dissolved in 68.5% of an aqueous solution of Paeonia suffruticosa flower (obtained through preparation according to step S103, in which the formulation ratio was the same as in Example 1), and the resulting solution was heated to 65° C. to obtain a phase C.

(4) The phase A and the phase B were mixed with stirring, and then the phase C was added for emulsification to obtain a coarse inclusion emulsion.

(5) The coarse emulsion was subjected to a high-pressure shearing treatment to achieve a nano-scale size, with a circulating water bath controlled at 30-40° C. and 6 cycles of shearing under a pressure of 800 bar, to obtain a whitening composition inclusion solution having a zeta potential of −29 mV, wherein the inclusion had a particle size of 106 nm. In Comparative Example 2, no co-emulsifier was added, such that the particle size of the inclusion became larger.

### Comparative Example 3: High-Pressure Shearing Treatment at 10-15° C.

(1) Based on mass ratio, 1% of glabridin was mixed with 20% of butylene glycol in a water bath at 65° C., and the mixture was stirred until complete dissolution was achieved to obtain a phase A.

(2) Based on mass ratio, 2% of polyglycerol-10 laurate, 1.5% of Paeonia suffruticosa seed oil, 5% of soybean lecithin, and 0.15% of palmitoyl tripeptide-8 were mixed in a water bath at 70° C., and the mixture was stirred until complete dissolution was achieved to obtain a phase B.

(3) Based on mass ratio, 1% of glutathione and 3% of 3-o-ethyl ascorbic acid were dissolved in 66.5% of an aqueous solution of Paeonia suffruticosa flower (obtained through preparation according to step S103, in which the formulation ratio was the same as in Example 1), and the resulting solution was heated to 65° C. to obtain a phase C.

(4) The phase A and the phase B were mixed with stirring, and then the phase C was added for emulsification to obtain a coarse inclusion emulsion.

(5) The coarse emulsion was subjected to a high-pressure shearing treatment to achieve a nano-scale size, with a circulating water bath controlled at 10-15° C. and 6 cycles of shearing under a pressure of 800 bar, to obtain a whitening composition inclusion solution having a zeta potential of −32 mV, wherein the inclusion had a particle size of 103 nm. In Comparative Example 3, the flexibility of the phospholipid bilayer membrane was reduced due to the lower temperature during the high-pressure shearing treatment, making it more difficult for the composition to be homogenized into a small-particle-size inclusion with uniform particle size. When the particle size distribution of the inclusion in the composition is wide, the vesicles with significantly different particle sizes tend to fuse due to Ostwald ripening, and with the growth of time or the intensification of Brownian motion caused by the increase in ambient temperature, the ripening ultimately causes most of the vesicles to aggregate and fuse, resulting in the phenomenon of stratification from the macroscopic appearance perspective.

### Comparative Example 4: Replacement of Palmitoyl Tripeptide-8 with Nonapeptide-1

(1) Based on mass ratio, 1% of glabridin was mixed with 20% of butylene glycol in a water bath at 65° C., and the mixture was stirred until complete dissolution was achieved to obtain a phase A.

(2) Based on mass ratio, 2% of polyglycerol-10 laurate, 1.5% of Paeonia suffruticosa seed oil, and 5% of soybean lecithin were mixed in a water bath at 70° C., and the mixture was stirred until complete dissolution was achieved to obtain a phase B.

(3) Based on mass ratio, 1% of glutathione, 3% of 3-o-ethyl ascorbic acid, and 0.02% of nonapeptide-1 were dissolved in 66.48% of an aqueous solution of Paeonia suffruticosa flower (obtained through preparation according to step S103, in which the formulation ratio was the same as in Example 1), and the resulting solution was heated to 65° C. to obtain a phase C.

(4) The phase A and the phase B were mixed with stirring, and then the phase C was added for emulsification to obtain a coarse inclusion emulsion.

(5) The coarse emulsion was subjected to a high-pressure shearing treatment to achieve a nano-scale size, with a circulating water bath controlled at 30-40° C. and 6 cycles of shearing under a pressure of 800 bar, to obtain a whitening composition inclusion solution having a zeta potential of −34 mV, wherein the inclusion had a particle size of 30 nm. In Comparative Example 4, palmitoyl tripeptide-8 was not added, and nonapeptide-1 having no amphiphilicity was used as the signal molecule (but nonapeptide-1 has an α-MSH antagonistic effect).

### Comparative Example 5: Replacement of Palmitoyl Tripeptide-8 with Palmitoyl Tetrapeptide-7

(1) Based on mass ratio, 1% of glabridin was mixed with 20% of butylene glycol in a water bath at 65° C., and the mixture was stirred until complete dissolution was achieved to obtain a phase A.

(2) Based on mass ratio, 2% of polyglycerol-10 laurate, 1.5% of Paeonia suffruticosa seed oil, 5% of soybean lecithin, and 0.06% of palmitoyl tetrapeptide-7 were mixed in a water bath at 70° C., and the mixture was stirred until complete dissolution was achieved to obtain a phase B.

(3) Based on mass ratio, 1% of glutathione and 3% of 3-O-ethyl ascorbic acid were dissolved in 66.44% of an aqueous solution of Paeonia suffruticosa flower (obtained through preparation according to step S103, in which the formulation ratio was the same as in Example 1), and the resulting solution was heated to 65° C. to obtain a phase C.

(4) The phase A and the phase B were mixed with stirring, and then the phase C was added for emulsification to obtain a coarse inclusion emulsion.

(5) The coarse emulsion was subjected to a high-pressure shearing treatment to achieve a nano-scale size, with a circulating water bath controlled at 30-40° C. and 6 cycles of shearing under a pressure of 800 bar, to obtain a whitening composition inclusion solution having a zeta potential of −28 mV, wherein the inclusion had a particle size of 38 nm. In Comparative Example 5, palmitoyl tripeptide-8 was not added, and palmitoyl tetrapeptide-7 having amphiphilicity was used as the signal molecule (but palmitoyl tetrapeptide-7 does not have an α-MSH antagonistic effect).

### Comparative Example 6: Replacement of Palmitoyl Tripeptide-8 with Palmitoyl Pentapeptide-4

(1) Based on mass ratio, 1% of glabridin was mixed with 20% of butylene glycol in a water bath at 65° C., and the mixture was stirred until complete dissolution was achieved to obtain a phase A.

(2) Based on mass ratio, 2% of polyglycerol-10 laurate, 1.5% of Paeonia suffruticosa seed oil, 0.06% of palmitoyl pentapeptide-4, and 5% of soybean lecithin were mixed in a water bath at 70° C., and the mixture was stirred until complete dissolution was achieved to obtain a phase B.

(3) Based on mass ratio, 1% of glutathione and 3% of 3-o-ethyl ascorbic acid were dissolved in 66.44% of an aqueous solution of Paeonia suffruticosa flower (obtained through preparation according to step S103, in which the formulation ratio was the same as in Example 1), and the resulting solution was heated to 65° C. to obtain a phase C.

(4) The phase A and the phase B were mixed with stirring, and then the phase C was added for emulsification to obtain a coarse inclusion emulsion.

(5) The coarse emulsion was subjected to a high-pressure shearing treatment to achieve a nano-scale size, with a circulating water bath controlled at 30-40° C. and 6 cycles of shearing under a pressure of 800 bar, to obtain a whitening composition inclusion solution having a zeta potential of −25 mV, wherein the inclusion had a particle size of 34 nm. In Comparative Example 6, palmitoyl tripeptide-8 was not added, and palmitoyl pentapeptide-4 having amphiphilicity was used as the signal molecule (but palmitoyl pentapeptide-4 does not have an α-MSH antagonistic effect).

### Comparative Example 7: Without Addition of Phospholipid

(1) Based on mass ratio, 1% of glabridin was mixed with 20% of butylene glycol in a water bath at 65° C., and the mixture was stirred until complete dissolution was achieved to obtain a phase A.

(2) Based on mass ratio, 6% of polyglycerol-10 laurate, 1.5% of Paeonia suffruticosa seed oil, and 0.15% of palmitoyl tripeptide-8 were mixed in a water bath at 70° C., and the mixture was stirred until complete dissolution was achieved to obtain a phase B.

(3) Based on mass ratio, 1% of glutathione and 3% of 3-O-ethyl ascorbic acid were dissolved in 67.35% of an aqueous solution of Rosa rugosa flower (obtained through preparation according to step S103, in which the formulation ratio was the same as in Example 1), and the resulting solution was heated to 65° C. to obtain a phase C.

(4) The phase A and the phase B were mixed with stirring, and then the phase C was added for emulsification to obtain a coarse inclusion emulsion.

(5) The coarse emulsion was subjected to a high-pressure shearing treatment to achieve a nano-scale size, with a circulating water bath controlled at 30-40° C. and 6 cycles of shearing under a pressure of 800 bar, to obtain a whitening composition inclusion solution having a zeta potential of −32 mV, wherein the inclusion had a particle size of 28 nm. In Comparative Example 7, no phospholipid was added, and no circular structure was formed in the whitening composition inclusion solution.

### Comparative Example 8: with Reference to Example 7 in CN115778886A

In S1, 6% of Glycyrrhiza glabra extract, 5% of apple extract, 2% of rice bran extract, 8% of ethylhexyl cocoate, 3% of diglyceryl stearate malate, 2% of isopropyl myristate, 4% of Beheneth-20, 4% of SORBETH-30, 2% of ethoxydiglycol, and 5% of 1,2-hexanediol were mixed and heated at 75° C. to obtain an oil phase; then, 5% propylene glycol and 5% sorbitol were added to 49% pure water, and the mixture was heated in a water bath at 75° C. for dissolution to obtain an aqueous phase.

In S2, the aqueous phase was added to the oil phase at a rate of 9 drops/s, and the mixture was mixed by stirring at 75° C. and 1000 rpm; after the mixing was completed, a high-speed shearing treatment was performed at a rotation speed of 15000 rpm for 3 min to obtain a micron-scale dispersion.

In S3, the micron-scale dispersion was subjected to 6 cycles of high-pressure homogenization treatment at 75° C. under a pressure of 800 bar to obtain the glabridin plant-derived micro inclusion.

### Comparative Example 9: With Reference to Example 1 in CN114796008A

In an argon atmosphere, 0.1 g of glabridin and 4 g of polyglyceryl-3-methylglucose distearate were dissolved in an oil phase containing 0.5 g of jojoba oil, 0.5 g of Limnanthes alba seed oil, and 3 g of dicaprylyl carbonate, and the formula amounts of pentylene glycol, glycerol, trehalose, sodium oleate, and water were weighed and mixed to make up to 100 g, serving as an aqueous phase after dissolution. After the two phases achieved complete dissolution, the oil phase was slowly added to the aqueous phases at 80° C. under shearing conditions, and then the mixture was subjected to 6 cycles of high-pressure homogenization at 600 bar and 60° C. to obtain a supramolecular composition nano solution.

### Comparative Example 10 to Comparative Example 11: Investigation of Signal Molecule

Comparative Example 10 followed the same conditions as Example 3 to obtain a whitening composition inclusion solution, except that the palmitoyl tripeptide-8 was replaced with nonapeptide-1.

Comparative Example 11 followed the same conditions as Example 3 to obtain a whitening composition inclusion solution, except that the palmitoyl tripeptide-8 was replaced with undecylenoyl phenylalanine.

### Comparative Example 12: Investigation of Auxiliary Ingredient

Comparative Example 12 followed the same conditions as Example 3 to obtain a whitening composition inclusion solution, except that the glutathione was replaced with cysteine.

### Comparative Example 13: Whitening Composition Inclusion Solution Containing 0% of Palmitoyl Tripeptide-8 and 1.15% of Glabridin

Comparative Example 13 followed the same conditions as Example 3 to obtain a whitening composition inclusion solution, except that the palmitoyl tripeptide-8 was removed and the feed amount of the glabridin was changed to 1.15%.

### Comparative Example 14: Whitening Composition Inclusion Solution Containing 0% of Glabridin and 1.15% of Palmitoyl Tripeptide-8

Comparative Example 14 followed the same conditions as Example 3 to obtain a whitening composition inclusion solution, except that the glabridin was removed and the feed mass percentage of the palmitoyl tripeptide-8 was changed to 1.15%.

### Comparative Example 15: Whitening Composition Inclusion Solution Containing 0% of Glutathione and 2% of Glabridin

Comparative Example 15 followed the same conditions as Example 3 to obtain a whitening composition inclusion solution, except that the glutathione was removed and the feed mass percentage of the glabridin was changed to 2%.

### Comparative Example 16: Whitening Composition Inclusion Solution Containing 0% of Glabridin and 2% of Glutathione

Comparative Example 16 followed the same conditions as Example 3 to obtain a whitening composition inclusion solution, except that the glabridin was removed and the feed mass percentage of the glutathione was changed to 2%.

### Experimental Example 1: Stability Test

After the whitening composition inclusion solutions of the above examples or comparative examples were separately left to stand at room temperature for 30 days, the particle size, polydispersity index, and appearance state of each of the whitening composition inclusion solutions were tested, and the results are shown in Table 1.

TABLE 1
Stability test results

| | | Particle size | Polydispersity | |
|---|---|---|---|---|
| Sample | Time | (nm) | index (PDI) | Appearance state |
| Example 1 | 30 days | 87 | 0.437 | High transparency and slight |
| | | | | blue tint, homogeneity, no |
| | | | | agglomeration and stratification |
| Example 2 | 30 days | 126 | 0.453 | High transparency and slight |
| | | | | blue tint, homogeneity, no |
| | | | | agglomeration and stratification |
| Example 3 | 30 days | 36 | 0.405 | High transparency and slight |
| | | | | blue tint, homogeneity, no |
| | | | | agglomeration and stratification |
| Comparative | 30 days | 33 | 0.398 | Homogeneity, no agglomeration |
| Example 1 | | | | and stratification |
| Comparative | 30 days | 116 | 0.587 | Slightly reduced transparency, |
| Example 2 | | | | slight precipitation of particles, |
| | | | | change in color to pink |
| Comparative | 30 days | 121 | 0.468 | Significantly reduced transparency, |
| Example 3 | | | | slight precipitation of particles, |
| | | | | change in color to pink |
| Comparative | 30 days | 31 | 0.396 | Homogeneity, no agglomeration |
| Example 4 | | | | and stratification |
| Comparative | 30 days | 40 | 0.412 | Homogeneity, no agglomeration |
| Example 5 | | | | and stratification |
| Comparative | 30 days | 38 | 0.429 | Homogeneity, no agglomeration |
| Example 6 | | | | and stratification |
| Comparative | 30 days | 29 | 0.387 | Homogeneity, no agglomeration |
| Example 7 | | | | and stratification |
| Comparative | 30 days | 76 | 0.458 | Slightly reduced transparency, |
| Example 8 | | | | change in color to pink, no |
| | | | | agglomeration and stratification |
| Comparative | 30 days | 94 | 0.386 | Homogeneity, no agglomeration |
| Example 9 | | | | and stratification |

From Table 1, it can be seen that: Comparative Examples 2 and 3 turned pink when glabridin was precipitated; the change in color to pink usually does not occur when the glabridin is stably encapsulated, so the results indicate that glabridin was stabilized by the encapsulations in the other groups.

From Table 1, it can be seen that: Comparative Examples 2 and 3 were not involved in the comparison due to their poor practicality, because they could not be stored for a long time.

### Experimental Example 2: Investigation of Whitening Effects

Investigation 1: In order to compare the whitening effects between the examples and the comparative examples, the Dermalab Combo and VISIA skin analyzers were used for efficacy testing of the samples, and the testing standard followed was Testing Method for Assessment of Cosmetic Whitening Efficacy, T/ZHCA 001-2018. Samples of 4% of Example 3, 4% of Comparative Example 1, 4% of Comparative Example 4, 4% of Comparative Example 5, 4% of Comparative Example 6, and 4% of Comparative Example 7 were each added to a blank essence, and the MI values and ITA° values of 30 volunteers aged 20 to 60 years were tested and compared before and after skin application using the above samples.

Investigation 2: Following the same procedures, samples of 4% of Example 3 and 4% of each of Comparative Examples 8-16 were each added to a blank essence, and the MI values and ITA° values of 30 volunteers aged 20 to 60 years were tested and compared before and after skin application using the above samples.

The lower the MI value, the less melanin in the skin, and the fairer the skin looks. The higher the ITA° value, the whiter and brighter the skin.

TABLE 2
Comparison of MI values

| | | The first | The first | The second | The third | The fourth |
|---|---|---|---|---|---|---|
| Test | | day | week | week | week | week |
| Investigation | Example 3 | 151 | 135 | 96 | 88 | 85 |
| 1 | Comparative | 151 | 140 | 112 | 109 | 102 |
| | Example 1 | | | | | |
| | Comparative | 151 | 141 | 108 | 96 | 95 |
| | Example 4 | | | | | |
| | Comparative | 151 | 141 | 119 | 112 | 105 |
| | Example 5 | | | | | |
| | Comparative | 151 | 143 | 121 | 115 | 107 |
| | Example 6 | | | | | |
| | Comparative | 151 | 138 | 106 | 101 | 98 |
| | Example 7 | | | | | |
| Investigation | Example 3 | 154 | 136 | 98 | 90 | 83 |
| 2 | Comparative | 154 | 149 | 138 | 136 | 132 |
| | Example 8 | | | | | |
| | Comparative | 154 | 145 | 136 | 127 | 112 |
| | Example 9 | | | | | |
| | Comparative | 154 | 139 | 121 | 115 | 101 |
| | Example 10 | | | | | |
| | Comparative | 154 | 140 | 109 | 99 | 97 |
| | Example 11 | | | | | |
| | Comparative | 154 | 139 | 105 | 99 | 95 |
| | Example 12 | | | | | |
| | Comparative | 154 | 134 | 127 | 119 | 98 |
| | Example 13 | | | | | |
| | Comparative | 154 | 151 | 148 | 142 | 145 |
| | Example 14 | | | | | |
| | Comparative | 154 | 132 | 95 | 93 | 98 |
| | Example 15 | | | | | |
| | Comparative | 154 | 150 | 142 | 139 | 135 |
| | Example 16 | | | | | |

TABLE 3
Comparison of ITA° values

| | | The first | The first | The second | The third | The fourth |
|---|---|---|---|---|---|---|
| Test | | day | week | week | week | week |
| Investigation | Example 3 | 24.6 | 25.1 | 25.8 | 26.3 | 27.3 |
| 1 | Comparative | 24.6 | 24.4 | 25.2 | 25.4 | 26.1 |
| | Example 1 | | | | | |
| | Comparative | 24.6 | 24.9 | 25.3 | 25.9 | 26.5 |
| | Example 4 | | | | | |
| | Comparative | 24.6 | 24.7 | 25.1 | 25.5 | 25.9 |
| | Example 5 | | | | | |
| | Comparative | 24.6 | 24.6 | 24.9 | 25.2 | 25.7 |
| | Example 6 | | | | | |
| | Comparative | 24.6 | 24.9 | 25.3 | 25.8 | 26.8 |
| | Example 7 | | | | | |
| Investigation | Example 3 | 24.8 | 25.4 | 25.9 | 26.4 | 27.5 |
| 2 | Comparative | 24.8 | 25.0 | 25.3 | 25.8 | 26.1 |
| | Example 8 | | | | | |
| | Comparative | 24.8 | 24.9 | 25.1 | 25.3 | 25.6 |
| | Example 9 | | | | | |
| | Comparative | 24.8 | 25.1 | 25.6 | 25.9 | 26.6 |
| | Example 10 | | | | | |
| | Comparative | 24.8 | 24.6 | 25.0 | 25.5 | 25.9 |
| | Example 11 | | | | | |
| | Comparative | 24.8 | 25.0 | 25.3 | 25.9 | 26.5 |
| | Example 12 | | | | | |
| | Comparative | 24.8 | 25.2 | 25.4 | 25.7 | 26.9 |
| | Example 13 | | | | | |
| | Comparative | 24.8 | 24.9 | 25.1 | 25.3 | 25.1 |
| | Example 14 | | | | | |
| | Comparative | 24.8 | 25.7 | 26.2 | 27.1 | 26.3 |
| | Example 15 | | | | | |
| | Comparative | 24.8 | 25.2 | 25.4 | 25.7 | 26.9 |
| | Example 16 | | | | | |

Conclusion: As shown in Table 2, the MI values (skin pigmentation) of Example 3, Comparative Example 1, and Comparative Examples 4-16 showed a significant decreasing trend with the time of use; among them, Example 3 demonstrated the most significant effect in reducing the skin melanin content (in the fourth week, the MI value decreased by 43.7%), which was far superior to Comparative Example 1 and Comparative Examples 4-16.

As shown in Table 3, the ITA° values (human skin color) of Example 3, Comparative Example 1, and Comparative Examples 4-16 showed a significant increasing trend with the time of use; among them, Example 3 demonstrated the most significant effect in enhancing the skin tone (in the fourth week, the ITA° value increased by 11.0%), which was far superior to Comparative Example 1 and Comparative Examples 4-16.

Traditionally, palmitoyl tripeptide-8 is used as a polypeptide for relieving and resisting allergy, rather than as a target peptide. The present application employs palmitoyl tripeptide-8 primarily as a target peptide and also considers its antagonistic effect. The MI value of Comparative Example 4 showed the second-largest decrease (in the fourth week, the MI value decreased by 37.1%, and the ITA° value increased by 7.7%); compared with Example 3, in Comparative Examples 4 and 10, the target peptide was replaced with nonapeptide-1, which also exhibits a very good match with the MC1 receptor on melanocytes, but has no amphiphilicity although antagonizing α-MSH, resulting in a relatively poor carrier-targeting effect. Comparative Examples 1/5/6 showed similar effects. Although Comparative Examples 5 and 6 adopted the target peptides with structures similar to that of example 3, neither palmitoyl tetrapeptide-7 nor palmitoyl pentapeptide-4 possess an α-MSH antagonistic effect, resulting in a poorer MI value-decreasing effect in Comparative Examples 5 and 6 than in Comparative Example 4. Comparative Example 1 did not adopt the target peptide, but the MI value-decreasing effect and ITA° value-increasing effect thereof were still superior to those of Comparative Examples 5 and 6. In summary, since the phospholipid vesicles can encapsulate both water-soluble and oil-soluble active ingredients, combined with the target peptide, they can improve the whitening effect.

The MI value of Comparative Example 7 showed the second-largest decrease (in the fourth week, the MI value decreased by 35.1%, and the ITA° value increased by 8.9%); compared with Example 3, in Comparative Example 7, no phospholipid was added, so that no circular structure (bilayer vesicle) was formed. In summary, since the phospholipid vesicles can encapsulate effective ingredients, combined with the target peptide, they can improve the whitening effect.

In addition, as can be seen from the results of Example 3 and Comparative Examples 8-16, when glabridin, palmitoyl tripeptide-8 and glutathione were used together, a synergy effect was exhibited therebetween, and changing any one of these three ingredients to another similar ingredient makes it difficult to achieve the technical effect of synergy, so that the technical effect of synergy achieved by the present invention is unexpected.

In order to further verify the synergy effect and the degree of synergy among the ingredients in the whitening composition inclusion solution of the present application, the applicant compared the in-vitro whitening efficacy of Example 3, Comparative Examples 17-19, and Test Examples 1-5. The specific procedures were as follows:

### Comparative Example 17

(1) Based on mass ratio, 0.04% of glabridin and 5% of butylene glycol were stirred in a water bath with the temperature maintained at 65° C. until complete dissolution was achieved to obtain a phase A.

(2) Based on mass ratio, 0.36% of hydroxypropyl-β-cyclodextrin was dissolved in 94.6% of water with stirring, and the solution was heated to 65° C. to obtain a phase B.

(3) The phase B was added dropwise and slowly to the phase A with continuous stirring, and the mixture was stirred with the temperature maintained at 65° C. until no visible particles were observed to obtain a phase C.

(4) Based on mass ratio, 0.4% of phenoxyethanol as a preservative was added to the phase C, and the mixture was stirred until complete dissolution was achieved to obtain water-soluble glabridin. The ratio simulated the addition of 4% of the whitening composition of Example 3 into a water-based skincare product, containing only the concentration of glabridin.

### Comparative Example 18

(1) Based on mass ratio, 0.006% of palmitoyl tripeptide-8 and 5% of butylene glycol were stirred at 65° C. until complete dissolution was achieved to obtain a phase A.

(2) Based on mass ratio, 0.04% of glutathione, 0.12% of 3-O-ethyl ascorbic acid, and 2.654% of an aqueous solution of Paeonia suffruticosa flower (obtained through preparation according to step S103, in which the formulation ratio was the same as in Example 1) were added into 91.78% of water, and the mixture was stirred at room temperature until complete dissolution was achieved to obtain a phase B.

(3) The phase A and the phase B were mixed, followed by addition of 0.4% of phenoxyethanol, and the mixture was stirred until uniform, resulting in a simulated addition of 4% of the whitening composition of Example 3 into a water-based skincare product, containing other active substances except for glabridin and their corresponding concentrations.

### Comparative Example 19

(1) Based on mass ratio, 0.04% of glabridin and 2.5% of butylene glycol were stirred in a water bath with the temperature maintained at 65° C. until complete dissolution was achieved to obtain a phase A.

(2) Based on mass ratio, 0.36% of hydroxypropyl-β-cyclodextrin was dissolved in 70% of water with stirring, and the solution was heated to 65° C. to obtain a phase B.

(3) The phase B was added dropwise and slowly to the phase A with continuous stirring, and the mixture was stirred with the temperature maintained at 65° C. until no visible particles were observed to obtain a phase C.

(4) Based on mass ratio, 0.006% of palmitoyl tripeptide-8 and 2.5% of butylene glycol were stirred at 65° C. until complete dissolution was achieved to obtain a phase D.

(5) Based on mass ratio, 0.04% of glutathione, 0.12% of 3-O-ethyl ascorbic acid, and 2.654% of an aqueous solution of Paeonia suffruticosa flower (obtained through preparation according to step S103, in which the formulation ratio was the same as in Example 1) were added into 21.38% of water, and the mixture was stirred at room temperature until complete dissolution was achieved to obtain a phase E.

(6) The phase D, the phase E, and the phase C were sequentially mixed, followed by addition of 0.4% of phenoxyethanol as a preservative, and the mixture was stirred until uniform, resulting in a simulated addition of 4% of the whitening composition of Example 3 into a water-based skincare product, containing active substances and their corresponding concentrations.

### Test Example 1: Diluted Test Sample of Example 1

Based on mass ratio, 4% of the whitening composition inclusion solution of Example 3 was dissolved in 91.4% of water, followed by addition of 4.2% of butylene glycol and 0.4% of phenoxyethanol, and the mixture was stirred uniformly to obtain a test sample simulating the addition of 4% of the whitening composition inclusion solution of Example 3 into a water-based skincare product.

### Test Example 2: Diluted Test Sample of Comparative Example 13

Based on mass ratio, 4% of the whitening composition inclusion solution of Comparative Example 13 was dissolved in 91.4% of water, followed by addition of 4.2% of butylene glycol and 0.4% of phenoxyethanol, and the mixture was stirred uniformly to obtain a test sample simulating the addition of 4% of the whitening composition inclusion solution of Comparative Example 13 into a water-based skincare product.

### Test Example 3: Diluted Test Sample of Comparative Example 14

Based on mass ratio, 4% of the whitening composition inclusion solution of Comparative Example 14 was dissolved in 91.4% of water, followed by addition of 4.2% of butylene glycol and 0.4% of phenoxyethanol, and the mixture was stirred uniformly to obtain a test sample simulating the addition of 4% of the whitening composition inclusion solution of Comparative Example 14 into a water-based skincare product.

### Test Example 4: Diluted Test Sample of Comparative Example 15

Based on mass ratio, 4% of the whitening composition inclusion solution of Comparative Example 15 was dissolved in 91.4% of water, followed by addition of 4.2% of butylene glycol and 0.4% of phenoxyethanol, and the mixture was stirred uniformly to obtain a test sample simulating the addition of 4% of the whitening composition inclusion solution of Comparative Example 15 into a water-based skincare product.

### Test Example 5: Diluted Test Sample of Comparative Example 16

Based on mass ratio, 4% of the whitening composition inclusion solution of Comparative Example 16 was dissolved in 91.4% of water, followed by addition of 4.2% of butylene glycol and 0.4% of phenoxyethanol, and the mixture was stirred uniformly to obtain a test sample simulating the addition of 4% of the whitening composition inclusion solution of Comparative Example 16 into a water-based skincare product.

### Experimental Example 3: In-Vitro Whitening Efficacy

Negative control: A blank sample was prepared with reference to the preparation method in Example 3, except that glabridin, palmitoyl tripeptide-8, and glutathione were removed, while the mass ratios of the remaining ingredients were the same as in Example 3; then, based on mass ratio, 4% of the blank sample was dissolved in 91.4% of water, followed by addition of 4.2% of butylene glycol and 0.4% of phenoxyethanol, and the mixture was stirred uniformly to obtain a negative control.

A 3D melanin skin model (MelaKutis®) was used as a testing tool to verify the synergy effect. Starting from the day the model was received (defined as day 0), UVB irradiation treatment (50 mJ/cm²) was performed daily. After continuous stimulation for 3 days, the samples of the positive control, Test Examples 1-5, Comparative Examples 17-19, and negative control were each systematically administered to the melanin model. The administration was performed daily, and after 6 days of continuous stimulation, the administration ended. At the same time, a blank control was set up (the L* value and melanin content of the model not subjected to UVB irradiation or drug administration, with the cultivation time and other cultivation conditions consistent with those in the test groups). The in-vitro whitening efficacy of the sample groups was evaluated by the luminance (L* value) and melanin distribution of the skin model after administration.

TABLE 4
Comparison of L* values and melanin contents

| | | Ratio of | | Ratio of |
|---|---|---|---|---|
| | | increase in | | decrease |
| | | L* value | | in melanin |
| | | compared to | | compared to |
| | | negative | Melanin | negative |
| | L* value | control | content | control |
| Blank control | 69.87 | / | 0.248 | / |
| Negative | 56.25 | / | 0.359 | / |
| control | | | | |
| Positive | 71.75 | 27.6% | 0.333 | 7.2% |
| control | | | | |
| Comparative | 62.1 | 10.4% | 0.347 | 3.3% |
| Example 17 | | | | |
| Comparative | 63.38 | 12.7% | 0.339 | 5.6% |
| Example 18 | | | | |
| Comparative | 69.02 | 22.7% | 0.268 | 25.3% |
| Example 19 | | | | |
| Test Example 1 | 74.39 | 32.2% | 0.221 | 38.4% |
| Test Example 2 | 65.89 | 17.1% | 0.318 | 11.4% |
| Test Example 3 | 65.59 | 16.6% | 0.326 | 9.2% |
| Test Example 4 | 66.28 | 17.8% | 0.306 | 14.8% |
| Test Example 5 | 64.75 | 15.1% | 0.334 | 7.0% |

As shown in Table 4, the L* value and melanin content of Comparative Example 19 were superior to those of Comparative Examples 17 and 18, indicating that compounding glabridin with the palmitoyl tripeptide-8, glutathione, vitamin C or vitamin C derivative, and aqueous plant solution exhibited a synergy effect. The results of Test Example 1 were all superior to those of Comparative Example 19, indicating that the encapsulation using the carrier form of the present invention also had an excellent permeation-enhancing effect.

The results of Test Example 1 were significantly superior to those of Test Examples 2-5 (which had the same total active ingredients as Test Example 1 but each lacked one active ingredient), indicating that glabridin had an unexpected synergy effect with palmitoyl tripeptide-8 and glutathione.

In conclusion, the present application adopted the compounding of glabridin and glutathione, supplemented with the antioxidant vitamin C or vitamin C derivative and the anti-inflammatory aqueous plant extract solution (refer to the data of Example 3 and Comparative Examples 1 and 7), which had already exerted a good synergistic effect, achieving a relatively good whitening effect; meanwhile, through the strict selection of target peptides, the targeting effect of the effective ingredients (glabridin, glutathione, etc.) was enhanced, so that the whitening effect was further improved. Palmitoyl tripeptide-8 played a relatively remarkable role in improving the whitening composition inclusion solution. The compounding of glabridin, glutathione, and palmitoyl tripeptide-8 exhibited the technical effect of significant synergy effect.

The above description is only for the purpose of illustrating the preferred embodiments of the present invention, and is not intended to limit the scope of the present invention. Any slight modifications, equivalent replacements, and improvements made to the above examples in accordance with the technical spirit of the present invention shall be included in the protection scope of the technical solutions of the present invention.

What is claimed is:

1. A preparation method for a nano-scale bilayer inclusion, comprising the following specific steps:
S1, weighing 1-3% of an alcohol-soluble main ingredient and 10-50% of a polyol by mass percentage, mixing the same in a water bath at 65° C., and stirring until complete dissolution is achieved to obtain a phase A;
S2, weighing 3-15% of a phospholipid, 1-3% of a co-emulsifier, 1-5% of a liquid oil, and 0.01-0.5% of a signal molecule by mass percentage, mixing the same in a water bath at 70° C., and stirring until complete dissolution is achieved to obtain a phase B;
S3, preparing an aqueous plant extract solution, then weighing 0.01-2% of an auxiliary ingredient and 0.01-4% of an antioxidant ingredient by mass percentage, followed by dissolving in 17.5-83.97% of the aqueous plant extract solution, and heating to 65° C. to obtain a phase C;
S4, mixing the phase A with the phase B, followed by stirring uniformly, and then adding the phase C for emulsification to obtain a coarse emulsion; and
S5, subjecting the coarse emulsion to a high-pressure shearing treatment at a temperature of 30-40° C. to obtain a nano-scale inclusion, thus ultimately obtaining a whitening composition inclusion solution.

2. The preparation method for the nano-scale bilayer inclusion according to claim 1, wherein in step S4, an emulsifying machine is used with an output power of 0.2 kW, a rotation speed of 3000-8000 rpm, and a high-speed shearing dispersion time of 3-10 min;
the shearing is performed at a pressure of 400-1000 bar, and the number of cycles of shearing is 5-10.

3. The preparation method for the nano-scale bilayer inclusion according to claim 1, wherein the aqueous plant extract solution is an aqueous extract solution of a herbal anti-inflammatory plant, the herbal anti-inflammatory plant being one or a combination of two or more of Paeonia suffruticosa flower, Paeonia suffruticosa root, Rosa rugosa flower, and Portulaca oleracea.

4. The preparation method for the nano-scale bilayer inclusion according to claim 3, wherein in step S3, the herbal anti-inflammatory plant is washed, dried, and crushed to obtain a plant powder, and the plant powder is decocted with water in a mass ratio of 1:10-1:20 under reflux at 50-80° C. for 8-24 h to obtain a decoction solution; then the decoction solution is filtered, and a filtrate is treated with a macroporous anion exchange resin in a volume ratio of (10-15):1, with a retention time of 15-25 min, to obtain a purified aqueous plant extract solution.

5. The preparation method for the nano-scale bilayer inclusion according to claim 1, wherein the alcohol-soluble main ingredient is glabridin, the auxiliary ingredient is glutathione, and the signal molecule is palmitoyl tripeptide-8.

6. The preparation method for the nano-scale bilayer inclusion according to claim 1, wherein the polyol is one or a combination of two or more of glycerol, propylene glycol, butylene glycol, dipropylene glycol, pentylene glycol, and isoprene glycol;
the co-emulsifier is one or a combination of two or more of polyglycerol-10 stearate, polyglyceryl-10 laurate, polyglyceryl-10 oleate, ethoxylated hydrogenated castor oil, and potassium cetyl phosphate;
the liquid oil is one or a combination of two or more of caprylic/capric triglyceride, Limnanthes alba seed oil, squalane, olive oil, and Paeonia suffruticosa seed oil;
the phospholipid is one or a combination of two or more of soybean lecithin, hydrogenated lecithin, and soybean phosphatidylcholine;
the antioxidant ingredient is one or a combination of two or more of vitamin C, 3-o-ethyl ascorbic acid, ascorbyl glucoside, magnesium ascorbyl phosphate, sodium ascorbyl phosphate, and ascorbic acid polypeptide.

7. The preparation method for the nano-scale bilayer inclusion according to claim 6, wherein a formulation ratio of the whitening composition inclusion solution is: 1.1±0.1% of glabridin, 20±5% of the polyol, 6±2% of the phospholipid, 2±0.5% of the co-emulsifier, 2±0.5% of the liquid oil, 0.15±0.05% of palmitoyl tripeptide-8, 1±0.2% of glutathione, 3±0.5% of the antioxidant ingredient, and 60±15% of the aqueous plant extract solution by mass percentage; and/or
the nano-scale bilayer inclusion has a particle size of 20-110 nm or 30-110 nm.

8. A high-transparency and stable whitening composition inclusion solution prepared by using the preparation method for the nano-scale bilayer inclusion according to claim 7, wherein the phospholipid serves as a main wall material of the inclusion, and the alcohol-soluble main ingredient, the auxiliary ingredient, the antioxidant ingredient, and the plant extract comprising an anti-inflammatory ingredient are encapsulated in the inclusion, with the signal molecule attached to the surface of the inclusion.

9. Use of the whitening composition inclusion solution according to claim 8 in the field of cosmetics.

10. A whitening composition inclusion solution, having a preparation method comprising:
(1) based on mass ratio, mixing 1% of glabridin with 20% of butylene glycol in a water bath at 65° C., and stirring until complete dissolution is achieved to obtain a phase A;
(2) based on mass ratio, mixing 2% of polyglycerol-10 laurate, 1.5% of Paeonia suffruticosa seed oil, 5% of soybean lecithin, and 0.15% of palmitoyl tripeptide-8 in a water bath at 70° C., and stirring until complete dissolution is achieved to obtain a phase B;
(3) based on mass ratio, dissolving 1% of glutathione and 3% of 3-O-ethyl ascorbic acid in 66.35% of an aqueous solution of Rosa rugosa flower, and heating to 65° C. to obtain a phase C,
wherein the preparation of the aqueous solution of Rosa rugosa flower comprising: washing, drying, and crushing Rosa rugosa flower as a herbal anti-inflammatory plant to obtain a plant powder, decocting the plant powder with water in a mass ratio of 1:15 under reflux at 60° C. for 8 h to obtain a decoction solution, then filtering the decoction solution, and treating a filtrate with a macroporous anion exchange resin in a volume ratio of 10:1, with a retention time of 20 min, to obtain a purified aqueous plant extract solution;
(4) mixing the phase A and the phase B with stirring, and then adding the phase C for emulsification to obtain a coarse inclusion emulsion; and
(5) subjecting the coarse emulsion to a high-pressure shearing treatment to achieve a nano-scale size, with a circulating water bath controlled at 30-40° C. and 6 cycles of shearing under a pressure of 800 bar, to obtain the whitening composition inclusion solution,
wherein optionally, the whitening composition inclusion solution has a zeta potential of −34 mV;
optionally, the inclusion in the whitening composition inclusion solution has a particle size of 30 nm.
